(19) United States
(12) Patent Application Publication
(10) Pub. No.: US 2025/0099159 A1
(43) Pub. Date: Mar. 27, 2025

(54) SYSTEM AND METHOD FOR ABLATING A TISSUE SITE BY ELECTROPORATION WITH REAL-TIME PULSE MONITORING

(71) Applicant: AngioDynamics, Inc., Latham, NY (US)

(72) Inventor: Robert NEAL, II, Richmond, VA (US)

(73) Assignee: AngioDynamics, Inc., Latham, NY (US)

(21) Appl. No.: 18/905,204

(22) Filed: Oct. 3, 2024

Related U.S. Application Data
(63) Continuation of application No. 14/837,480, filed on Aug. 27, 2015, now Pat. No. 12,114,911.
(60) Provisional application No. 62/042,862, filed on Aug. 28, 2014.

(51) Int. Cl. A61B 18/12; A61B 17/00; A61B 18/00; A61B 18/14

(52) U.S. Cl. CPC A61B 18/1233; A61B 18/1206; A61B 18/14; A61B 2017/00115; A61B 2017/00119; A61B 2017/00154; A61B 2018/00577; A61B 2018/00613; A61B 2018/00642; A61B 2018/00702; A61B 2018/00708; A61B 2018/00767

(57) ABSTRACT

A medical system for ablating a tissue site by electroporation with real-time pulse monitoring during an electroporation treatment procedure is provided. The treatment control module monitors applied pulses and detect an abnormal condition that indicates either an ineffective electroporation of the tissue or an imminent excessive current such as a spark event that may exceed the capacity of the pulse generator. Rather than terminating the procedure entirety, the treatment control module temporarily pauses the procedure to allow the user to adjust the pulse parameters so as to complete the procedure without starting the procedure over again.

### CROSS REFERENCE TO RELATED APPLICATIONS

This application is a continuation of co-pending U.S. patent application Ser. No. 14/837,480, filed Aug. 27, 2015, published 20160058493 A1, which claims the benefit of priority from U.S. Provisional Application No. 62/042,862, filed Aug. 28, 2014, each of which is hereby incorporated herein by reference.

### FIELD OF THE INVENTION

The present invention relates to a control system for controlling an electroporation medical treatment device and more particularly, to such devices with real-time monitoring of pulse delivery during an electroporation treatment procedure.

### BACKGROUND OF THE INVENTION

Medical devices for delivering therapeutic energy such as electrical pulses to tissue include one or more electrodes and a pulse generator. The pulse generator allows the electrode to deliver the therapeutic energy to a targeted tissue, thereby causing ablation of the tissue.

Electroporation procedure parameters that influence the size and shape of their affected region include the nature of the tissue (cellularity, extracellular constituent composition, anisotropy, conductivity, metabolic demand), the pulse delivery apparatus (number of electrodes, their size, and relative geometry), and pulse parameters (voltage, number of pulses, pulse length, pulse delivery rate). In addition to the above, the generator's maximum pulse intensity capabilities (maximum voltage and current) dictate the maximum achievable treatment region. Where controllable and large lesions are desired, it is important to maintain pulses that are capable of inducing electroporation effects to the tissue while remaining below the maximum generator capacity.

In conventional electroporation devices, before the treatment procedure a physician would decide on a particular pulse delivery apparatus and select the pulse parameters. Once the treatment procedure starts, the electroporation device follows the pre-treatment programming set by the physician and delivers the pulses according to the pre-selected pulse parameters. Specifically, there was no way to alter the settings other than to stop the procedure manually. Even when the procedure completes normally1 there was no assurance that there were clinically sufficient electroporation of the targeted region due to the unpredictable nature of patient environments and living tissue.

Moreover, under certain conditions, the electroporation device can produce an unwanted spark across the electrodes, which causes the pulse generator to freeze or shut down prematurely. As a result, the treatment procedure would have to be restarted from the beginning.

Therefore, it would be desirable to provide a system and method for monitoring the electroporation pulse delivery in real time and to alter the treatment parameters based on the monitoring as the treatment progresses and without the necessity to repeat the entire procedure.

### SUMMARY OF THE DISCLOSURE

According to one aspect of the present invention, a medical system for ablating a tissue site by electroporation with real-time pulse monitoring during an electroporation treatment procedure is provided. The system includes electrodes, a pulse generator and a treatment control module, The pulse generator is configured to generate and apply between the electrodes multiple electroporation pulses according to selected pulse parameters for ablation of tissue in the target region. The treatment control module detects an abnormal condition of the applied electrical pulses in real-time during an electroporation treatment procedure and temporarily pauses the procedure without termination when the abnormal condition is detected. The abnormal condition indicates that a predetermined current threshold value is about to be exceeded or an ineffective electroporation of the tissue. Advantageously, the pause allows a physician to adjust the pulse parameters so as to complete the procedure without starting the procedure over again.

According to another aspect of the present invention, a method of ablating a tissue site by electroporation with real-time pulse monitoring during an electroporation treatment procedure is provided.

In an electroporation treatment procedure, at least two electrodes are positioned near a target region of a tissue site of a patient and multiple electroporation pulses are applied between the electrodes according to selected pulse parameters for ablation of tissue in the target region. During the application of pulses, sensed values of the applied pulses are continuously received and monitored in real-time as the treatment procedure progresses. When an abnormal condition is detected based on the continuously received values of the applied pulses while the electroporation treatment procedure is progressing, the treatment procedure Is temporarily paused without termination. The abnormal condition indicates either an ineffective electroporation of the tissue or an imminent excessive current such as a spark event that may exceed the capacity of the pulse generator. Rather than terminating the procedure entirely, the procedure is temporarily paused to allow the physician to adjust the pulse parameters so as to complete the procedure without starting the procedure over again.

### BRIEF DESCRIPTION OF THE DRAWINGS

FIG. 1 is a functional block diagram of an electroporation device according to one aspect of the present invention.

FIG. 2 is a functional block diagram of a treatment control computer of FIG. 1.

FIG. 3 is a functional block diagram of a pulse generator shown in FIG. 1

FIG. 4 is a functional block diagram of a sensor of FIG. 3.

FIG. 5A is a flowchart of a method of ablating a tissue site by electroporation with real-time pulse parameter monitoring during an electroporation procedure.

FIG. 5B is a flowchart of a method of detecting an abnormal condition of delivered electrical pulses.

FIG. 6 illustrates an expected electrical pulse,

FIGS. 7A and 7B illustrate an electrical pulse having an absolute high current and absolute low current, respectively.

FIGS. 8A and 8B illustrate electrical pulses having no expected rise in current.

FIG. 9 illustrates an electrical pulse having a current creep.

FIGS. 10A and 10B illustrate an electrical pulse having an unstable current.

FIG. 11 is an exemplary screenshot of a recommended parameter change presentation.

### DETAILED DESCRIPTION OF THE INVENTION

Throughout the present teachings, any and all of the one, two, or more features and/or components disclosed or suggested herein, explicitly or implicitly, may be practiced and/or implemented in any combinations of two, three, or more thereof, whenever and wherever appropriate as understood by one of ordinary skill in the art. The various features and/or components disclosed herein are all illustrative for the underlying concepts, and thus are non-limiting to their actual descriptions. Any means for achieving substantially the same functions are considered as foreseeable alternatives and equivalents, and are thus fully described in writing and fully enabled. The various examples, illustrations, and embodiments described herein are by no means, in any degree or extent, limiting the broadest scopes of the claimed inventions presented herein or in any future applications claiming priority to the instant application.

The present invention uses real-time electrical pulse monitoring during electroporation to control input settings and procedure parameters to optimize the electroporation procedure. Namely, during the electroporation procedure, the system monitors pulse parameters to determine an abnormal condition of the applied electrical pulses and whether changes should be made to the input parameters such as pulse voltage, pulse duration, pulse rate and the like. The abnormal conditions being monitored include a possibility of an imminent spark or ineffective electroporation. When an abnormal condition has been detected; the system can either pause pulse delivery to allow the physician to change the settings or automatically change one or more of the pulse parameters without user input. Once the parameter has been changed, the system then resumes the pulse delivery based on the changed settings. By doing this, electroporation pulse delivery can be optimized by maintaining effective strength pulses without exceeding pre-determined generator limits or causing errors.

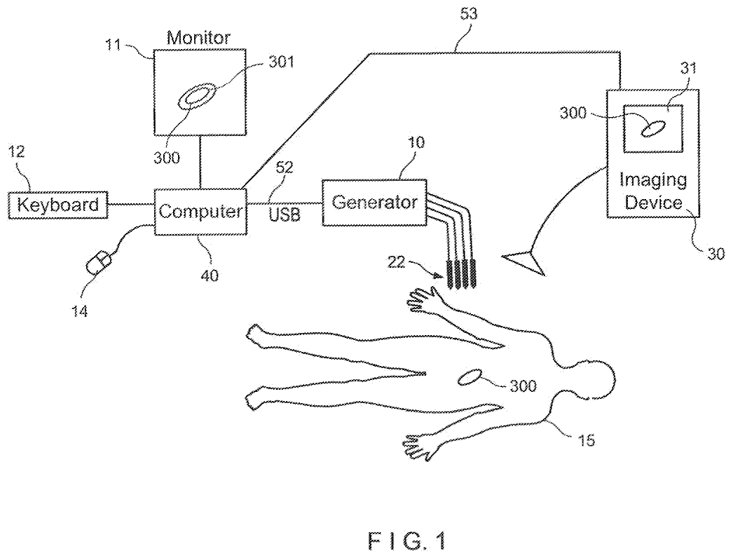

One embodiment of the present invention is illustrated in FIG. 1. The components used with the present invention are illustrated in FIG. 1. One or more electrodes/probes 22 deliver therapeutic energy and are powered by a voltage pulse generator 10 that generates high voltage puls.es as therapeutic energy such as pulses capable of irreversibly electroporating the tissue cells. In the embodiment shown, the voltage pulse generator 10 includes six separate receptacles for receiving up to six individual probes 22 which are adapted to be plugged into the respective receptacle. The receptacles are each labeled with a number in consecutive order. In other embodiments, the voltage pulse generator 10 can have any number of receptacles for receiving more or less than six probes.

Each probe 22 includes either a monopolar electrode, bipolar electrodes having two electrodes separated by an insulating sleeve, or multipolar electrodes having greater than two electrode surfaces separated by an insulating sleeve which can be energized simultaneously or at different times. In one embodiment, if the probe includes a monopolar electrode, the amount of exposure of the active portion of the electrode can be adjusted by retracting or advancing an insulating sleeve relative to the electrode. See, for example, U.S. Pat. No. 7,344,533, which is incorporated by reference herein. In the embodiment shown, the probes 22 are monopolar electrodes. The generator 10 is connected to a treatment control computer 40 having input devices such as keyboard 12 and a pointing device 14, and an output device such as a display device 11 for viewing an image of a target treatment area such as a lesion 300 surrounded by a safety margin 301. The therapeutic energy delivery device 20 is used to treat a lesion 300 inside a patient 15. An imaging device 30 includes a monitor 31 for viewing the lesion 300 inside the patient 15 in real time. Examples of imaging devices 30 include ultrasonic, CT, MRI and fluoroscopic devices as are known in the art.

For purposes of this application, the terms “code”, “software”, “program”, “application” “software code”, “software module”, “module” and “software program” are used interchangeably to mean software instructions that are executable by a processor.

The “user” can be a physician or other medical professional. The treatment control module 54 executed by a processor outputs various data including text and graphical data to the monitor 11 associated with the generator 10.

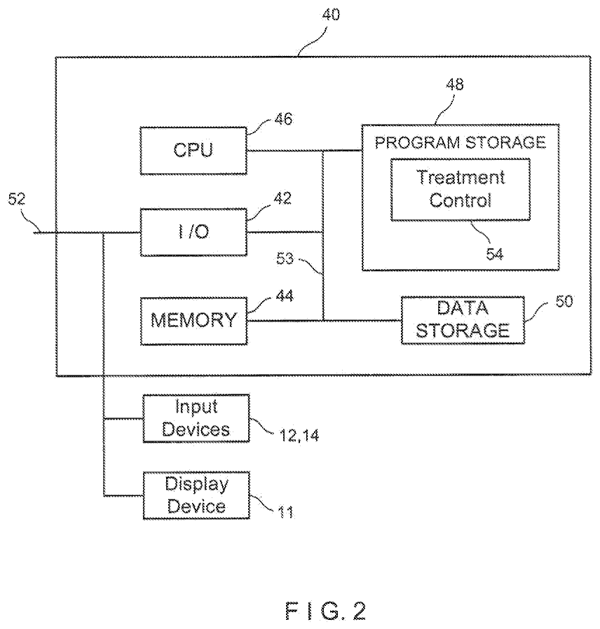

Referring now to FIG. 2, the treatment control computer 40 of the present invention is connected to the communication link 52 through an I/O interface 42 such as a USB (universal serial bus) interface, which receives information from and sends information over the communication link 52 to the voltage generator 10. The computer 40 includes memory storage 44 such as RAM, processor (CPU} 46, program storage 48 such as ROM or EEPROM, and data storage 50 such as a hard disk, all commonly connected to each other through a bus 53. The program storage 48 stores, among others, computer software (treatment control module 54) which assists a user/physician to plan for, execute, and review the results of a medical treatment procedure, The treatment control module 54, executed by the processor 46, assists a user to plan for a medical treatment procedure by enabling a user to more accurately position each of the probes 22 of the therapeutic energy delivery device 20 in relation to the lesion 300 in a way that will generate the most effective treatment zone. The treatment control module 54 can display the anticipated treatment zone based on the position of the probes and the treatment parameters. The treatment control module 54 can display the progress of the treatment in real time and can display the results of the treatment procedure after it is completed. This information can be used to determine whether the treatment was successful and whether it is necessary to re-treat the patient.

The treatment control module 54 is also adapted to detect in real-time any abnormal condition of the pulses while they are being delivered during an electroporation treatment procedure. If any abnormal condition is detected, the treatment control module 54 is programmed to pause the procedure wm1out terminating the procedure. The pause allows a physician to change one or more of the pulse parameters through the module's user interface and to continue the procedure based on the changed parameters without terminating the procedure.

The program storage 48 stores a database/table of recommended pulse parameter changes for each of the abnormal conditions being checked by the treatment control module 54. When the pulses are being delivered and an abnormal condition is detected, the user interface portion of the control module 54 retrieves the recommended parameter changes from the database for the particular abnormal condition and presents them to the user through the display 11, The treatment control module 54 can also change criteria for detecting the abnormal condition based on initial treatment pulse parameters programmed by the user. For example, different body parts/organs or different health/age of patients may require different thresholds as their conductivity and susceptibility to irreversible electroporation may differ. Thus, for example, the absolute high current threshold value can be changed depending on the tissue type to be treated. The control module 54 may then store the changed threshold value in the program storage 48 for later use as the new criteria.

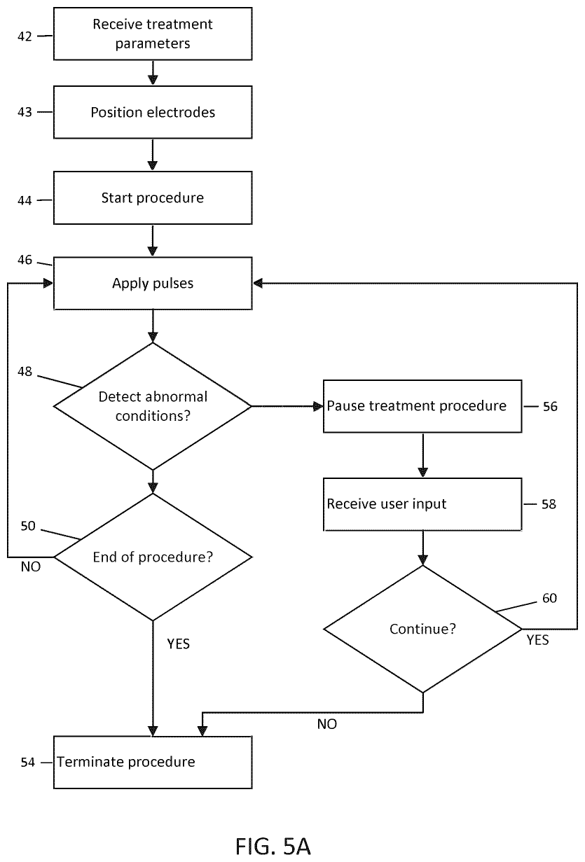

A more detailed discussion of the control module 54 will be made later herein with reference to FIGS. 5A and 5B.

Any of the software program modules in the program storage 48 and data from the data storage 50 can be transferred to the memory 44 as needed and is executed by the CPU 46.

In one embodiment, the computer 40 is built into the voltage generator 10. In another embodiment, the computer 40 is a separate unit which is connected to the voltage generator through the communications link 52, The communication link 52 can be, for example, a USB link.

In one embodiment, the imaging device 30 is a stand alone device which is not connected to the computer 40. In the embodiment as shown in FIG. 1, the computer 40 is connected to the imaging device 30 through a communications link 53. As shown, the communication link 53 is a USB link. In this embodiment, the computer can determine the size and orientation of the lesion 300 by analyzing the data such as the image data received from the imaging device 30, and the computer 40 can display this information on the monitor 11. In this embodiment, the lesion image generated by the imaging device 30 can be directly displayed on the grid 200 of the monitor 11 of the computer running the treatment control module 54. This embodiment would provide an accurate representation of the lesion image on the grid 200, and may eliminate the step of manually inputting the dimensions of the lesion in order to create the lesion image on the grid 200. This embodiment would also be useful to provide an accurate representation of the lesion image if the lesion has an irregular shape.

It should be noted that the software can be used independently of the generator 10. For example, the user can plan the treatment in a different computer as will be explained below and then save the treatment parameters to an external memory device, such as a USB flash drive (not shown). The data from the memory device relating to the treatment parameters can then be downloaded into the computer 40 to be used with the generator 10 for treatment.

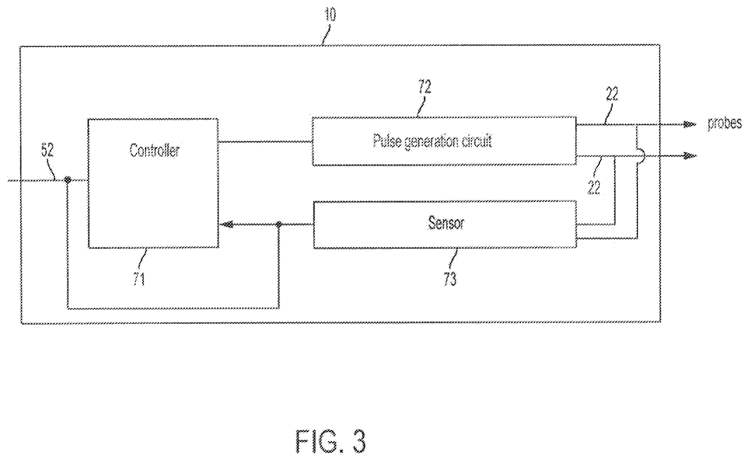

FIG. 3 is a functional block diagram of a pulse generator 10 shown in FIG. 1. FIG. 2 illustrates one embodiment of a circuitry to detect an abnormality in the applied pulses. The abnormal condition for the applied pulses includes an absolute high current, absolute low current, current creep and unstable current A USS connection 52 carries instructions from the user computer 40 to a controller 71. The controller 71 can be a computer similar to the computer 40 as shown in FIG. 2. The controller 71 can include a processor, ASIC (application-specific integrated circuit), microcontroller or wired logic. The controller 71 then sends the instructions to a pulse generation circuit 72. The pulse generation circuit 72 generates the pulses and sends electrical energy to the probes. For clarity, only one pair of probes/electrodes are shown. However, the generator 10 can accommodate any number of probes/electrodes such as 6 probes. In the embodiment shown, the pulses are applied one pair of electrodes at a time, and then switched to another pair. The pulse generation circuit 72 includes a switch, preferably an electronic switch, that switches the probe pairs based on the instructions received from the controller 71.

A sensor 73 can sense the current and voltage between each pair of the probes in real time and communicate such information to the controller 71, which in turn, communicates the information to the computer 40. Although the treatment control module 54 houses the software code for detecting an abnormal condition of the applied pulses, it may be beneficial for the controller 71 to store such detection module as certain abnormal conditions occur quite rapidly and the speed of detection is important and in some cases critical Accordingly, the abnormal condition detection module can be implemented in hardware as a customized wired logic as part of the controller 71.

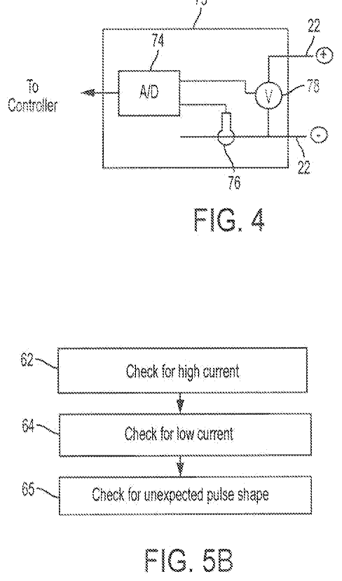

FIG. 4 is a functional block diagram of a sensor 73 of FIG. 3. The sensor 73 includes a voltage sensor 78 connected across a pair of electrodes 22 and a current sensor 76 connected to a negative electrode (return conduit) in the pair of electrodes. Although FIGS. 3-4 show two electrodes from two wires 22, there may be multiple electrodes between two wires 22. The sensed values are continuously received and digitized by an A/D converter 74 and transmitted to the controller 71. Preferably, the A/D converter 74 can sample the sensed values at a very fast rate and preferably at a rate of at least 100 MHz (100 million samples per second) for the control module 54 to be able to react to the abnormal conditions very quickly.

The current sensor 76 can be a Hall effect sensor/probe which is positioned around an electrode so as to measure the electric current without directly interfering with the pulse signal. Typically, the current sensor 76 is placed on the negative signal connection of the electrode pair. If the electrode pairs are switched, then only one current sensor connected at the input side of the switch is needed. Otherwise, if there are 3 pairs of electrodes, for example, and all are firing at the same time, there will be 3 current sensors so as to measure the electric current of each pair separately. In that case, the current from the three sensors will need to be added.

The voltage sensor 78 can be a conventional voltage divider, comprised of two serially connected resistors, that measures a voltage drop across a known resistance value.

The voltage sensor 78 uses resistors which are of much higher resistance than the tissue (kΩ MΩ, versus tissue, which is hundreds of Ω), and thus induces negligible effect on the strength of the pulses delivered to the tissue. A correction factor is calculated for the divider circuit based on the resistances of the two resistors in the voltage divider circuit and the resistance of the load (tissue resistance) to determine the true delivered voltage to the tissue based on the measured voltage drop across the resistor.

A method of ablating a tissue site by electroporation with real-time pulse parameter monitoring during an electroporation procedure will now be explained with reference to FIGS. 5A-5B.

The steps executed are part of the treatment control module 54 which can be part of the computer 40 or a part of the controller 71 in the pulse generator 10 itself for faster response. Referring to FIG. 5A, in step 42, the treatment control module 54 graphically interacts with the user to receive treatment parameters which include voltage between electrodes, electrode separation distance, firing sequence among the electrode pairs, pulse delivery/firing rate, pulse duty cycle, number of pulses in a pulse set/train, number of pulse sets/trains, inter-pulse delay, inter-pulse train delay, electrode exposure length, pulse parameter changes for each abnormal condition and the like. In step 43, a user/physician positions the electrodes 22 at a tissue site such that the electroporation field covers the target region. The target region is now ready to be treated.

In step 44, based on the received parameters, the pulse control module 54 instructs the controller 71 in the pulse generator 10 to start an electroporation procedure. In step 46, under the control of the controller 71, the pulse generation circuit 72 starts delivering electroporation pulses through the electrodes 22 that have been placed in the patient

In step 48, while the pulses are being delivered, the treatment control module 54 continuously receives the voltage and current values from the sensors 76 and 78 and monitors the received values in real-time for a series of abnormal conditions which may indicate a potential problem in effective delivery of pulses. The abnormal conditions to be detected will be discussed in more detail later with reference to FIG. 5B.

If an abnormal condition is detected, control passes to step 56, in which the treatment procedure is paused. It is important to note that the procedure is merely paused and the computer 40 remembers all of the pulse parameters and the point at which the procedure is stopped so that the procedure can be resumed where it paused.

As discussed above, the computer 40 stores in the storage 50 various recommended pulse parameter changes for each abnormal condition to be detected. Once the treatment procedure is paused, the treatment control module 54 in step 58 retrieves the recommended parameter changes and presents them to the user in the display. An exemplary screenshot of the presentation is shown in FIG. 11. FIG. 11 shows that the control module 54 has detected a high current risk and displayed the suggested parameter changes which have been retrieved from the storage 50. Specifically, the recommended parameter changes shown are a 10% reduction in voltage, 40% reduction in pulse length, and reduction of pulse rate to 60 pulses per minute. Typically, the recommended parameter changes are voltage reduction of 5-10%, and 30-50% reduction in pulse length.

The user has a choice of accepting the recommended changes, changing the recommended changes and aborting the electroporation procedure. Acceptance can be submitted by clicking on the ACCEPT button. Changing the recommended changes involves clicking the CANCEL button, inputting his own changes and then clicking the ACCEPT button, Aborting the procedure is done by simply clicking on ABORT button.

In step 60, the control module 54 determines whether the treatment procedure should be resumed based on the user input from step 58. If the answer is yes, then control passes to step 46 and the pulse generation circuit 72 resumes the treatment procedure with delivery of pulses based on the adjusted parameters. ff the answer is no, then control passes to step 54 which terminates the treatment procedure without completion.

At step 48, if no abnormal conditions were detected, control passes to step 50. At step 50, the control module 54 determines whether the delivery of pulses has been completed, if the answer is no, then control passes to step 46 and delivery of pulses continues. If the answer is yes, then control passes to step 54 which completes and terminates the treatment procedure normally.

FIG. 5B is a flowchart of a method of detecting an abnormal condition of delivered electrical pulses, which describes step 48 of FIG. 5A in more detail. Step 62 detects whether the current of the delivered pulses reaches a high current threshold value. As shown in FIG. 7A, the high current threshold value I_{H} can be 50 amps. When the delivered pulse reaches 50 amps at any time during the pulse, the abnormal condition is triggered.

Step 64 detects whether the delivered pulses have an absolute low current below a low current threshold value I_{L}. As shown in FIG. 7B, the low current threshold value can be 5 amps. When the delivered pulse reaches 5 amps or below at any time during the pulse, the abnormal condition is triggered. If the waveform is flat, it may indicate an insignificant or sub-optimal electroporation, and thus energy delivery should be increased, for example, by 10%.

Step 65 detects whether the delivered pulses have an unexpected pulse shape in order to predict a potential problem in a system shutdown or ineffective ablation. When this condition is detected, it indicates that a predetermined current threshold value (e.g., 50 amps) is about to be exceeded.

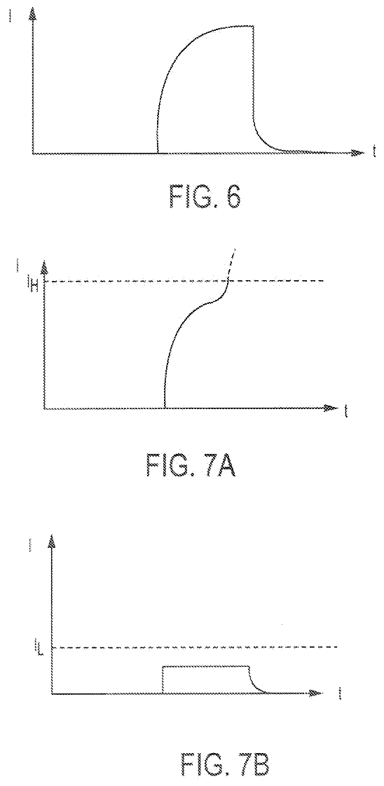

To monitor and detect unexpected pulse shapes, it may be useful to understand what an expected pulse should look like. Due to the progressive electroporation of tissue cells and a small rise in temperature within the delivery of an electroporation pulse, electrical conductivity of the tissue rises. Consequently, electrical currents typically rise in a decaying fashion within the pulse (approaching a plateau) as shown by FIG. 6, for example.

Similarly, between successive pulses, the current also expectedly rises due to the rise in tissue conductivity. An absence of corresponding current rise over a given number of pulses may serve as an indicator of insufficient energy intensity, and thus induce the need to increase pulse parameter strength.

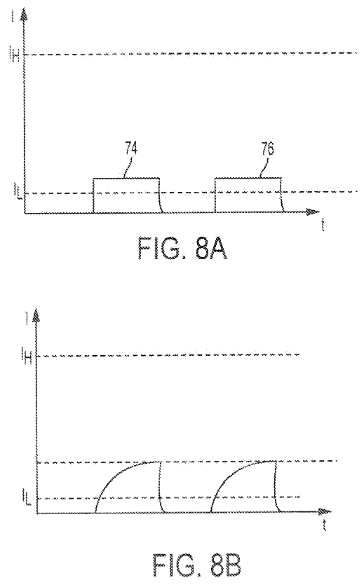

Thus, one of the unexpected pulse shapes is a stable waveform with very little increase (e.g., less than 5% threshold increase) or no increase in current either within a pulse or between pulses. As shown in FIG. 8A, each of pulses 74 and 76 has substantially no intra-pulse or inter-pulse current rise. In one embodiment, there is substantially no inter-pulse or intra-pulse rise if the rise is 5% or less. The control module 54 detects either condition as an unexpected pulse/waveform shape.

FIG. 8B illustrates another example of an inter-pulse stable waveforms. Even though each waveform 78, 80 has an expected rise in current, the change in current between the two pulses is virtually zero. In that case, the control module 54 detects the condition as an unexpected pulse shape.

One way to implement the steady intra-pulse waveform as shown in FIG. 8A is to take two measurements near the leading and falling edges (e.g., measurements at t=5 μsec and t=95 μsec for a 100 μsec pulse) and compare against a predetermined threshold current value (e.g., 5%). If the current difference is less than 5% over the measurement taken at t=5, then the pulse is considered to have an unexpected pulse shape.

Similarly, one way to implement the steady inter-pulse waveform as shown in FIG. 8B is to take two measurements near the falling edges of the pulses 78, 80 (e.g., measurements at t=95 μsec for a 100 μsec pulse) and compare against a predetermined threshold current value (e.g., 5%). If the current difference is less than 5% over the measurement taken at t=95 μsec for waveform 78, then the pulse is considered to have an unexpected pulse shape.

Another one of the unexpected pulse shapes is due to a condition called the “current creep”. This is one of the most common phenomena in electroporation pulse delivery which causes issues for clinical (and pre-clinical) applications.

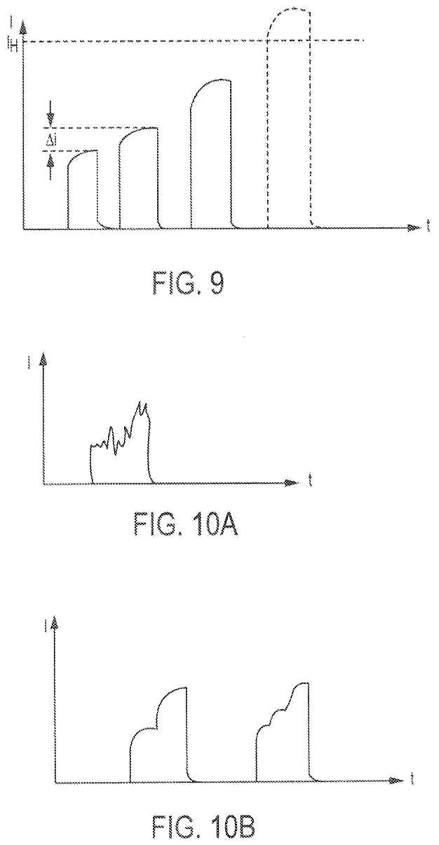

Normally, successive pulses are expected to have a slightly higher current because as electroporation progresses, the conductivity of tissue rises due to electroporation and because the temperature tends to increase. However, an excessive rise in current indicates an “abnormal current creep” as shown in FIG. 9 that could lead to the subsequent pulses to exceed the maximum current value I_{H}. Alternatively, an abnormal current creep can include a condition where the current creep can be slow, but the current value is near the maximum current value I_{H}.

For example, let's assume that the electroporation treatment procedure involves a 100 pulse protocol with 10 sets of pulses with 10 pulses in each set. If the average current for sets 1 through 4 are respectively 30 amps, 35 amps, 42 amps and 48 amps, then there is a high probability that the next set will exceed the maximum high current threshold I_{H} of 50 amps. Sometimes an abnormal current creep can be subtle such as 45, 45.5, 46 and then 48 amps. Because, 48 amps is near the maximum current value I_{H} of 50 amps, this may be a cause for concern. In these cases, the control module at step 65 detects the current creep as an abnormal condition in real-time before the fifth set of pulses is delivered.

There are several methods to implement the detection of an abnormal current creep. A first method is to measure the maximum current in each pulse and examine the difference between two successive pulse currents. If the difference is above a threshold difference current value, e.g., predetermined threshold rate of 7 amps, then that means the current is rising too fast. This situation may trigger the abnormal condition.

A second method is to calculate the above value or a 3-5 point moving average of current values (measured at the maximum current value in each pulse) or use a regression analysis to predict the next current value. The current values can be continuously sampled within a pulse (e.g. 100 sampling points per pulse) and the highest current value for each pulse can be used for the moving average and regression analysis.

If the predicted current value is higher than the maximum current threshold I_{H}, it will trigger the abnormal condition. For example, a 3 point moving average can be used and its slope can be examined to see if the next pulse might cause the current to exceed the maximum current threshold value. If so, the abnormal condition can be triggered.

The first and second methods above can be combined so that the unexpected pulse shape (and thus an abnormal condition) is triggered when both conditions are satisfied. In other words, if the difference between two successive pulse currents is above the threshold difference current value and the maximum current of the latest delivered pulse is above the maximum high current threshold value I_{H}, then the abnormal condition (abnormal current creep) is triggered by the control module 54.

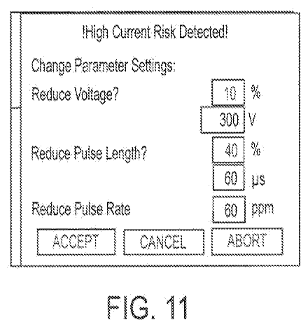

Another one of the unexpected pulse shapes is an unstable waveform as illustrated in FIG. 10A. It is not uncommon for baseline tissue properties or their changes in response to electroporation pulses to cause highly unstable and unpredictable waveforms. This can result in sudden spikes in current that may exceed safe or generator-limited conditions.

One method to implement the unstable waveform condition is to sample the current value of 100 data points within a single delivered pulse, calculate a standard deviation and comparing it to a threshold instability criteria, e.g., threshold deviation value of 10 amps.

The standard deviation method can be combined with a maximum current threshold value I_{H}, e.g., 50 amps, as described with reference to FIG. 7A, so that the abnormal condition is triggered when both conditions are satisfied.

Yet another one of the unexpected pulse shapes is a multi-tiered waveform as shown in FIG. 10B. This is a step-wise sudden and sharp increase in electrical current during a pulse. This can even occur in a repeated and thus semi-predictable manner. It most commonly occurs in a two phase system, but can have multiple “tiers” within the pulse. Since these waveforms pose a risk to continued pulse delivery, the best approach is to reduce the voltage or pulse length (or cease pulse delivery among that pair of electrodes) as will be explained later herein. Again, the detection of an unstable waveform can be combined with a maximum threshold current value I_{H}, e.g., 40 amps, so that the abnormal condition is triggered when both conditions are satisfied.

As discussed above, real-time detection of an abnormal condition that may lead to a sudden spark across the electrodes or ineffective treatment allows the present invention to pause the treatment procedure to allow the user to adjust the pulse parameters to mitigate such risks without having to terminate the procedure.

If IRE pulses are biphasic pulses, the absolute value of each biphasic pulse can be compared from one pulse to the next, or if orientation of polarity alters the current behavior, then pulses of the same orientation can be compared.

The above disclosure is intended to be illustrative and not exhaustive. This description will suggest many modifications, variations, and alternatives may be made by ordinary skill in this art without departing from the scope of the invention. Those familiar with the art may recognize other equivalents to the specific embodiments described herein. Accordingly, the scope of the invention is not limited to the foregoing specification.

1-36. (canceled)

37. A system comprising:
a processor;
a generator coupled to the processor and to be operatively coupled to at least two electrodes, at least one of the at least two electrodes to be placed in a target region of a tissue site; and
a memory coupled to the processor, the memory comprising program instructions to perform operations comprising:
generating a plurality of electrical pulses between the at least two electrodes;
receiving an indication of at least one treatment parameter;
detecting an abnormal condition of an irreversible electroporation (IRE) treatment procedure based in part on the indication of the at least one treatment parameter;
pausing generation of at least one of the plurality of electrical pulses in response to detection of the abnormal condition;
receiving an indication to resume the IRE treatment procedure; and
resuming the IRE treatment procedure including resuming generation of the at least one of the plurality of electrical pulses to complete the IRE treatment procedure.

38. The system of claim 37, wherein the treatment parameter is a voltage value.

39. The system of claim 38, wherein the abnormal condition includes the voltage value exceeding a predetermined threshold.

40. The system of claim 39, wherein the operations further comprise reducing a voltage of the plurality of electrical pulses in response to the detection of the abnormal condition.

41. The system of claim 40, wherein the voltage is reduced by 5-10%

42. The system of claim 37, wherein the treatment parameter is a current value.

43. The system of claim 42, wherein the abnormal condition includes the current value exceeding a predetermined threshold.

44. The system of claim 42, wherein the abnormal condition includes the current value being below a predetermined threshold.

45. The system of claim 44, wherein the abnormal condition is insufficient to provide an effective ablation.

46. The system of claim 37, further comprising a sensor.

47. The system of claim 46, wherein the indication of the at least one treatment parameter is received from the sensor.

48. The system of claim 46, wherein the sensor is a current sensor or voltage sensor.

49. A system comprising:
a processor;
a generator coupled to the processor and to be operatively coupled to at least two electrodes, at least one of the at least two electrodes to be placed in a target region of a tissue site;
a sensor; and
a memory coupled to the processor, the memory comprising program instructions to perform operations comprising:
generating a plurality of electrical pulses between the at least two electrodes;
sensing at least one treatment parameter during the generation of the plurality of electrical pulses;
receiving an indication of the at least one treatment parameter;
detecting an abnormal condition of an irreversible electroporation (IRE) treatment procedure based in part on the indication of the at least one treatment parameter;
pausing generation of at least one of the plurality of electrical pulses in response to detection of the abnormal condition;
receiving an indication to resume the IRE treatment procedure; and
resuming the IRE treatment procedure including resuming generation of the at least one of the plurality of electrical pulses to complete the IRE treatment procedure.

50. The system of claim 49, wherein the treatment parameter is a voltage value.

51. The system of claim 50, wherein the abnormal condition includes the voltage value exceeding a predetermined threshold.

52. The system of claim 51, wherein the operations further comprise reducing a voltage of the plurality of electrical pulses in response to the detection of the abnormal condition.

53. The system of claim 49, wherein the treatment parameter is a current value.

54. The system of claim 53, wherein the abnormal condition includes the current value exceeding a predetermined threshold.

55. The system of claim 53, wherein the abnormal condition includes the current value being below a predetermined threshold.

56. The system of claim 55, wherein the abnormal condition is insufficient to provide an effective ablation.
